SEQUENCE LISTING (5 sequences)

SEQ ID NO: 1 - DNA, 22 bp, Artificial Sequence
  Description of Artificial Sequence Oligonucleotide with restriction site
attcatcgatataactaagtaa

SEQ ID NO: 2 - DNA, 70 bp, Artificial Sequence
  Description of Artificial Sequence Oligonucleotide with restriction site
aggccgcaggggccgcggccgcactagtggggatccttaattaactgcaggggccactggggcccggtac

SEQ ID NO: 3 - DNA, 70 bp, Artificial Sequence
  Description of Artificial Sequence Oligonucleotide with restriction site
cgggccccagtggcccctgcagttaattaaggatccccactagtgcggccgcggcccctgcggccttgca

SEQ ID NO: 4 - DNA, 71 bp, Artificial Sequence
  Description of Artificial Sequence Oligonucleotide with restriction site
agggccgcaggggccgcggccgcactagtggggatccttaattaagctgcagggccactggggcccggtac

SEQ ID NO: 5 - DNA, 71 bp, Artificial Sequence
  Description of Artificial Sequence Oligonucleotide with restriction site
cgggccccagtggccctgcagcttaattaaggatccccactagtgcggccgcggcccctgcggcccttgca

(19) United States
(12) Patent Application Publication
(10) Pub. No.: US 2004/0029223 A1
(43) Pub. Date: Feb. 12, 2004

(54) Bacterial two-hybrid system for protein interaction screening

(76) Inventors: Gouzel Karimova, Paris (FR); Daniel Ladani, Cachan (FR); Agnes Ullman, Paris (FR); Luc Selig, Charenton Le Pont (FR); Pierre Legrain, Paris (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/240,102

(22) PCT Filed: Mar. 29, 2001
(86) PCT No.: PCT/IB01/00773

(51) Int. Cl. C12P 21/06

(52) U.S. Cl. 435/69.1

(57) ABSTRACT

Two bacterial strains deficient in endogenous adenylate cyclase activity are provided. The strains, designated BTH101 and DHM1, are useful in a signal amplification system comprising a bacterial multi-hybrid system, and more preferably a two-hybrid system, of at least two chimeric polypeptides containing a first chimeric polypeptide corresponding to a first fragment of an enzyme and a second chimeric polypeptide corresponding to a second fragment of an enzyme or a modulating substance capable of activating said enzyme as described in published PCT application WO 99/28746. The signal amplification system is useful in a method of selecting a molecule of interest, which is capable of binding to target ligand, wherein the interaction between the molecule of interest and the target ligand is detected with the signal amplification system, and the kit therefor, as described in WO 99/28746. The strains are also useful in the method of screening for a substance capable of stimulating or inhibiting the interaction between a target ligand and a molecule of interest, and the kit therefor, as described in WO 99/28746.

### BACKGROUND OF THE INVENTION

The present invention concerns the construction of new strains useful in a method for selecting a molecule, a kit therefor, a method for screening a molecule, a kit therefor, and a signal amplification system comprising a bacterial multi-hybrid system. More particularly, the present invention relates to improvements to the technique referred as a multi-hybrid system described in PCT application No. WO 99/28746, published on Jun. 10, 1999. This technology is also described in Proc. Natl. Acad. Sci. USA in 1998, 95 (10) pages 5752-5756 (Karimora et al.).

A novel bacterial two-hybrid system that allows an easy in vivo screening and selection of functional interactions between two proteins has recently been described. This genetic system is based on the reconstitution of an artificial cAMP signal transduction pathway in an Escherichia coli adenylate cyclase deficient strain (cya). It takes advantage of the modular structure of the catalytic domain of Bordetella pertussis adenylate cyclase, which consists of two complementary fragments. Bordetella pertussis adenylate cyclase: a toxin with multiple talents Trends in Microbiol. 7, 172-176). When they are expressed separately in E. coli, they can not be converted to an active enzyme unless interacting polypeptides are genetically fused to these fragments.

In the bacterial two-hybrid system, interaction between the two chimeric proteins results in functional complementation between the two adenylate cyclase fragments and restoration of enzymatic activity. The resulting cAMP synthesis triggers the expression of several E. coli resident genes, thus giving rise to a selectable phenotype.

By design, this bacterial two-hybrid system is taking place in an E. coli cya strain, i.e., lacking its endogenous adenylate cyclase. Numerous E. coli cya strains harboring point mutations, deletions or insertions within the cya gene (adenylate cyclase structural gene) have been described. Using a modified phosphomycin selection procedure, the DHP1 strain was isolated. This strain is a spontaneous cya derivative of DH1 (F-, glnV44(AS), recA1, endA1, gyrA96 (Nal'), thil, hsdR17, spoT1, rfbD1). Functional complementation between hybrid proteins appeared to be much more efficient in DHB1 than in other cya strains that were tested, possibly, because of the higher stability of chimeric proteins.

It has been discovered, however, that DHP1 exhibits a high frequency (at about 10⁻⁶) of spontaneous reversion of the Cya⁻ phenotype towards a Cya⁺ phenotype. This characteristic is, therefore, limiting the utility of DHP1 in large scale screening applications, such as a library screening when a great number of transformed cells have to be analyzed.

There exists a need in the art for bacterial strains that can be employed in the methods and kits described in published PCT application No. 99/28746. The new bacterial strains should exhibit a Cya phenotype, allowing efficient functional complementation between standard hybrid proteins, and be suitable for use in large scale analysis of protein-protein interactions.

### SUMMARY OF THE INVENTION

This invention aids in fulfilling these needs in the art. More particularly, it is an aim of this invention to provide new tools for using the bacterial multi-hybrid systems, methods, and kits disclosed in published PCT application WO 99/28746.

More particularly, this invention relates to improvements in the methods and kits described in published PCT Application WO 99/28746. In particular, this invention provides improved bacterial strains for use in the methods and kits. The bacterial strains are selected from the group consisting of strain BTH101 having C.N.C.M. Deposit Accession No. I-2309 and strain DHM1 having C.N.C.M. Deposit Accession No. I2310.

In one embodiment, this invention provides a signal amplification system as described in WO 99/28746 using strain BTH101 or strain DHM1 of this invention.

In another embodiment, this invention provides a method of selecting a molecule of interest as described in WO 99/28746. The method utilizes the signal amplification system described in WO 99/28746 in which the strain BTH101 or the strain DHM1 is employed.

In a further embodiment, this invention provides a kit for selecting a molecule of interest. The kit is as described in WO 99/28746 and utilizes strain BTH101 or strain DHM1 of this invention.

This invention also provides a method of screening for a substance capable of stimulating or inhibiting the interaction between a target ligand and a molecule of interest as described in WO 99/28746. The method utilizes strain BTH101 or strain DEM1 of this invention.

Further, this invention provides a kit for screening for a substance capable of stimulating or inhibiting the interaction between a target ligand and a molecule of interest. The kit is as described in WO 99/28746 and utilizes strain BTH101 or strain DHM1 of this invention.

This invention also provides strain BTH101 having C.N.C.M. Deposit Accession No. I-2309.

In addition, this invention provides strain DHM1 having C.N.C.M. Deposit Accession No. I-2310.

In addition, the invention provides a molecule of interest identified by the method of the invention using the DHM1 and BTH101 strains. The molecule of interest can correspond to a polynucleotide capable of expressing a molecule, which interacts with a fused target ligand coupled with an enzyme or a fragment thereof.

This invention also provides a DNA library comprising a collection of vectors transformed in a bacterial multi-hybrid system of the invention using strain BTH101 or strain DHM1. Each plasmid can contain a polynucleotide coding for the molecule of interest fused to a polynucleotide coding for the first or the second fragment of an enzyme. The polynucleotide coding for the molecule of interest can be, for example, a fragment of genomic DNA of Helicobacter pylori.

In another embodiment, this invention provides an H. pylori DNA library, HGX BHP₃, having C.N.C.M. Deposit Accession No. I-2367.

In summary, the present invention relates to the use of new strains, which are characterized as DHM1 and BTH101, and plasmids contained in E. coli XL-1/pUT18, XL-1/pUT18C, XL1-1/pT25, and XL-1/pKT25.

### BRIEF DESCRIPTION OF THE DRAWINGS

FIG. 1 shows the results of zip-zip interaction in different E. coli cya strains. β-galactosidase activity is shown for three different strains, DHM1, BHT101, and DHP1.

FIG. 2 shows the results of interaction in different strains. β-galactosidase activity is shown for the strains DHM1 and BTH101.

FIG. 3 is a map of plasmid pUT18C NewSfi.

FIG. 4 is a map of plasmid pKT25 NewSfi.

### DETAILED DESCRIPTION OF THE INVENTION

Most biological processes involve specific protein-protein interactions. General methodologies to identify interacting proteins or to study these interactions have been extensively developed. Among them, the yeast two-hybrid system currently represents the most powerful in vivo approach to screen for polypeptides that could bind to a given target protein. It utilizes hybrid genes to detect protein-protein interactions by means of direct activation of a reporter-gene expression.

In essence, the two putative protein partners are genetically fused to the DNA-binding domain of a transcription factor and to a transcriptional activation domain, respectively. A productive interaction between the two proteins of interest will bring the transcriptional activation domain in the proximity of the DNA-binding domain and will trigger directly the transcription of an adjacent reporter gene (usually lacZ or a nutritional marker) giving a screenable phenotype. There is evidence that the transcription can be activated through the use of two functional domains of a transcription factor: a domain that recognizes and binds to a specific site on the DNA and a domain that is necessary for activation.

Published PCT application WO 99/28746 describes a signal amplification system comprising a bacterial multi-hybrid system of at least two chimeric polypeptides containing:

(a) a first chimeric polypeptide corresponding to a first fragment of an enzyme;

(b) a second chimeric polypeptide corresponding to a second fragment of an enzyme or a modulating substance capable of activating said enzyme, wherein the first fragment is fused to a molecule of interest and the second fragment or the modulating substance is fused to a target ligand, and wherein the activity of the enzyme is restored by the in vivo interaction between the said molecule of interest and the said target ligand and wherein a signal amplification is generated.

The signal amplification system is useful in a method of selecting a molecule of interest, which is capable of binding to a target ligand, wherein the interaction between the molecule of interest and the target ligand is detected with the signal amplification system by generating a signal amplification and triggering transcriptional activation.

Published PCT application WO 99/28746 also describes a kit for selecting a molecule of interest, wherein the kit comprises:

(a) the signal amplification system;

(b) an E. coli strain or in any bacterial strain deficient in endogenous adenylate cyclase or any other eukaryotic cell; and

(c) a medium allowing the detection of the complementation. The medium is selected from the group consisting of indicator or selective medium as minimal medium supplemented with lactose or maltose as unique carbon source, medium with antibiotics, medium to visualize fluorescence, conventional medium, and medium that allows the sorting by the presence of the phage receptor.

The kit described in WO 99/28746 for selecting the molecule of interest can also comprise:

(a) the signal amplification system, wherein the molecule of interest is a mutant molecule compared to the known wild type molecule;

(b) the signal amplification system, wherein the molecule of interest is the known wild type molecule as the control;

(c) E. coli strain or in any bacterial strain deficient in endogenous adenylate cyclase or any other eukaryotic cell;

(d) a medium allowing the detection of the complementation selected from the group consisting of indicator or selective medium as minimal medium supplemented with lactose or maltose as unique carbon source, medium with antibiotics, medium to visualize fluorescence, conventional medium, and medium that allows the sorting by the presence of the phage receptor for each signal amplification system; and

(e) means for detecting whether the signal amplification system with the mutant molecule is enhanced or inhibited with respect to the signal amplification system with wild type.

In addition, published PCT application WO 99/28746 describes a method of screening for a substance capable of stimulating or inhibiting the interaction between a target ligand and a molecule of interest. The stimulating or the inhibiting activity is detected with the signal amplification system according by means of generating an amplification and respectively of triggering or of abolishing transcriptional activation. The signal amplification and the triggered or abolished transcriptional activation are compared with those obtained from an identical signal amplification system without any substance. The method of screening for a substance capable of stimulating or inhibiting the interaction between a target ligand and a molecule of interest can be performed in an E. coil strain or in any bacterial strain deficient in endogenous adenylate cyclase or any other eukaryotic cell.

Further, published PCT application WO 99/28746 describes a kit for screening for the substance capable of stimulating or inhibiting the interaction between a target ligand and a molecule of interest. The kit comprises:

(a) the signal amplification system with the substance capable of stimulating or inhibiting the interaction between a target ligand and a molecule of interest;

(b) the signal amplification system without any substance as the control;

(c) an E. coli strain or any bacterial strain deficient in endogenous adenylate cyclase or any other eukaryotic cell;

(d) a medium allowing the detection of the complementation selected from the group consisting of indicator plate or selective medium as minimal medium supplemented with lactose or maltose as unique carbon source, medium with antibiotics, medium to visualize fluorescence, conventional medium, and medium that allows the sorting by the presence of the phage receptor, and

(e) means for detecting whether the signal amplification system with the substance is enhanced or inhibited with respect to the signal amplification system without any substance.

The original Escherichia coil cya strain, DHP1, used in the bacterial two-hybrid system and other methods and kits described in published PCT application WO 99/28746 was a spontaneous cya mutant that was isolated as phosphomycin resistant Although complementation worked efficiently in the DHP 1 strain, it had a tendency to revert to a Cya⁺ phenotype at a high frequency, which precluded its utilization in large scale screening.

New cya strains have been constructed. These new stains of this invention (i) should not revert and (ii) should exhibit an efficient complementation. Two such strains were selected, DHM1 and BTH101, and are described below. Bacterial strains and plasmids used are listed in Table 1.

TABLE 1
Bacterial strains and plasmids used in the study.

| | | Reference |
|---|---|---|
| Strain or plasmid | Relevant feature (s) | or source |
| Strains: | | |
| DH1 | F-, recA1, endA1, rfbD1, gyrA96 | E. coli Genetic |
| | (Nal^{r}), thi1, hsdR17, spoT1, | StockCenter |
| | glnV44 (AS) | |
| FB8 | F-, prototrophic | 1 |
| KL800 | Hfr secA215, fhuA21, lacY1, glnV44 | E. coli Genetic |
| | (AS), rfbD1, thi-1 | StockCenter |
| MC1061 | F-, araD139, Δ(araA-leu)7697, | E. coli Genetic |
| | Δ(codBlac1)3, galK16, galE15, | StockCenter |
| | mcrAO, relA1, rpsL150(Str^{r}), spoT1, | |
| | mcrB1, hsdR2 | |
| DHP1 | DH1,cya | 2 |
| DHP11 | DHP1, cya-854, ilv-691::Tn10 | This study |
| DHM1 | DHP11, cya-854 | This study |
| BTH99 | MC1061, cya-99 | This study |
| BTH100 | BTH99, lac⁺ | This study |
| BTH101 | BTH100, leu⁺ | This study |
| Plasmids | | |
| pRecA | Co1EI replication origin; harbors the | 3 |
| | recA gene; Amp^{R} | |
| pUC4K | Co1EI replication origin; Amp^{R} Kan^{R} | Pharmacia |
| pSKBluescript II | Co1EI replication origin; Amp^{R} | Stratagen |
| pSKDKancya | derivative of pSKBluescript II; har- | This study |
| | bors a part of the cyaA gene and | |
| | Kan-cassette from pUC4K; Kan^{R,} | |
| | Amp^{R} | |

1. Uzan, M. and Danchin A., 1978. Correlation between the serine sensitivity and the derepressibility of the ilv genes in Escherichia coli relA-mutants. Mol Gen Genet., 165:21-30.

2. Karimova, G., Pidoux, J., Ullmann, A. & Ladant, D., 1988. A bacterial two-hybrid system based on reconstituted signal transduction pathway. Proc. Natl. Acad. Sci. U.S.A. 95:5752-5756.

3. Sancar, A., Rupp, W. D., 1979. Physical map of the recA gene. Proc. Natl. Acad. Sci. U.S.A. 76:3144-3148.

The two strains of this invention, DHM1 and BTH101, can be employed in the methods and kits described in published PCT application WO 99/28746. The entire disclosure of published PCT application WO 99/28746 is relied upon and incorporated by reference herein. The terms used herein have the same definitions as in WO 99/28746.

The prior bacterial strain DHP1 and the new strains DHM1 and BTH101 will now be described in greater detail.

1. DHP1

DHP1 strain described in published PCT application WO 99/28746 is an adenylate cyclase deficient (cya) derivative of DH1 (F-, glnV44(AS), recA1, endA1, gyrA96, (Nal'), thil, hsdR17, spoT1, rfbD1) (25), and was isolated using phosphomycin as a selection antibiotic (Alper, M. D. & Ames, B. N. (1978) J. Bacteriol. 133, 149-57). Growth media used were the rich medium LB or the synthetic medium M63 (Miller, J. H. (1972) Experiments in molecular genetics (Cold Spring Harbor Laboratory, Cold Spring Harbor, N.Y.)) supplemented with 1% carbon source. Antibiotic concentrations were ampicillin 100 mg/ml and chloramphenicol 30 mg/ml. Screening for the ability to ferment sugars was performed either on MacConkey agar plates containing 1% maltose, or on LB plates containing 40 mg/ml X-Gal (5-Bromo-4-chloro-3-indolyl-β-D-galactopyranoside) and 0.5 mM IPTG (Isopropyl-β-D-thiogalactopyranoside).

2. DHM1: F-, reCa, endA1, gyrA96Nal'), thi1, hsdR17, spoT1, rfbD1, glnV44(AS), cya-854

DHM1 strain is a derivative of DHP1 that harbors a small deletion within the cya gene (cya-854) that was introduced by P1 transduction (Miller, 1972). For this purpose, a P1 vir phage lysate was prepared on the donor strain FB8 cya-854 ilv-691::Tn10 (kind gift of M. Perrote). This strain has a transposon Tn10 insert in close linkage to cya (Wanner, 1986). This linkage was used to transfer the cya-85s4 mutation into DHP1 that harbors a plasmid, pRecA, a pBR322 derivative that encodes the recA gene of E. coli and restores a Rec⁺ phenotype (G. Karimova, unpublished results). The resulting TcR (tetracycline resistant) transductants were tested for the presence of the mutant allele cya-854, a 200-base-pair deletion within the cya gene (Glaser, P., Roy A, Danchin, A., 1989). This was carried out by restreaking the cells on MacConkey/maltose indicator plates with tetracycline (25 μg/ml). The transductants harboring the cya-854 mutation could be readily distinguished from those harboring the original cya mutation from DHP1 as they do not revert (that is no Cya⁺ cells—red phenotype—could be detected in patches of a large number of colonies), whereas the transductants with the original DHP1 cya mutation reverted with high frequency as indicated by the appearance of red Cya⁺ cells within patches of a large number of colonies. The frequency of co-transduction of two markers, ilv-691::Tn10 and cya-854, was about 30%. One such trnsductant was selected and called DHP11.

DEM1 strain was constructed by P1 transduction of the wild type allele of ilv from E. coli K-12 FB8 into DHP11 harboring pRecA. The transductants were selected on minimal medium without added amino acids. After reisolation of the resulting transductants, the presence of the cya-857 mutation and the loss of Tn10 (Tc^{S}-sensitivity) were verified by plating on MacConkey/maltose and LB containing Tc (25 μg/ml) plates. To cure pRecA plasmid, the transductants DHM1(pRecA) were grown in L-broth overnight and then spread on MacConkey/maltose plates. To identify DHM1 cells that lost pRecA, 100 singles colonies were restreaked on the two plates: one LB plate containing ampicillin (Amp, 100 μg/ml) and one MacConkey/maltose plate without antibiotic. Plates were grown for 24 hours at 37° C. Several Amp-sensitive clones were picked from the MacConkey/maltose plate, and after restreaking and further testing for complementation, one clone, called DHM1, was selected.

To examine the frequency of Lac⁺ and Mal⁺ revertants among DHM1 cells, an overnight culture of DHM1 (10 ml) in LB was collected by centrifugation, washed 3 times by B63 medium, and plated on minimal medium with X-gal supplemented by maltose or lactose as unique carbon sources. Frequencies of Lac⁺ and Mal⁺ clones were, respectively, 10⁻⁷ and 10⁻⁹. Note that these Lac⁺ and Mal⁺ revertants are not Cya⁺ revertants but arise from cAMP/CAP independent mutations within the lac promoter or cAMP-independent CAP mutations (CRP*).

3. BTH101: F, galE15, ga1K16, rpsL1 (Str'), hsdR2, mcrA1, mcrB1, cya-99

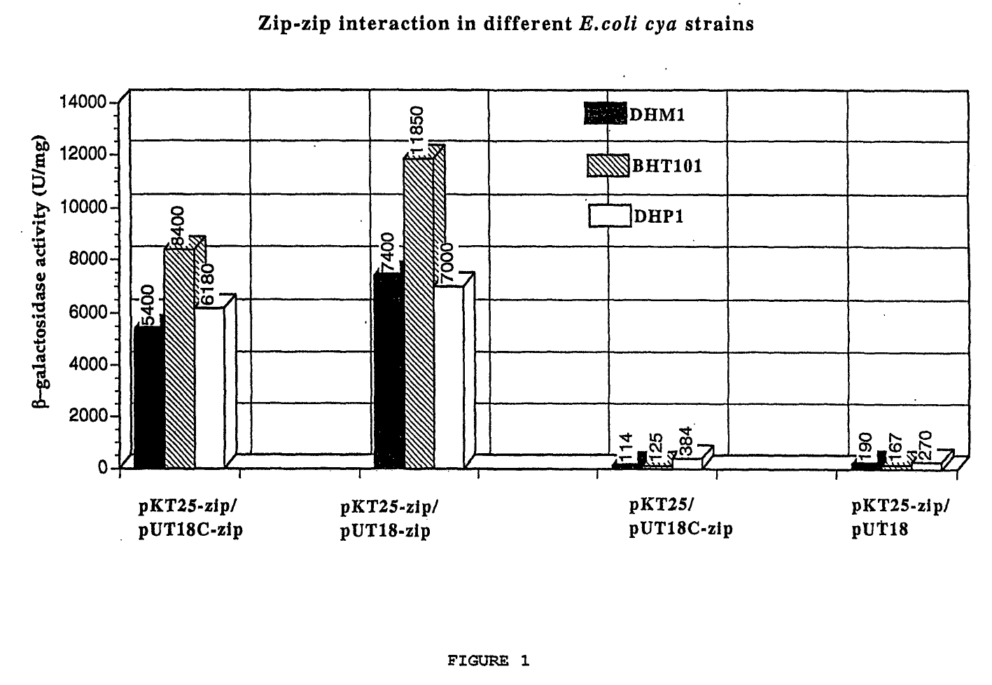

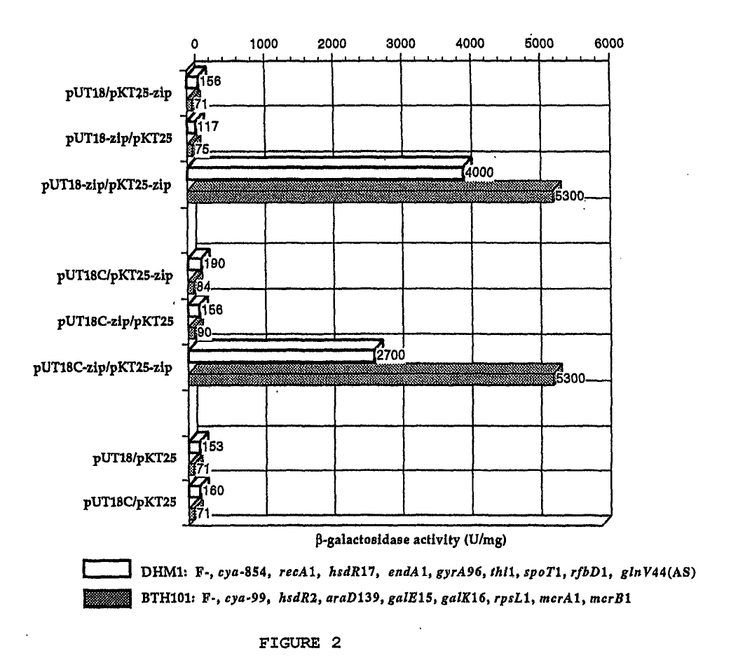

BTH101 strain is a derivative of MC1061 that was constructed by conjugation and P1 transduction techniques (Miller, 1972). While an attempt was made to introduce a precise deletion within the cya gene, we isolated a spontaneous cya mutant of MC1061, BTH99, that most likely harbors a deletion within the cya gene as it reverts to Cya⁺ (in fact Lac⁺ or Mal⁺ phenotype) with a very low frequency. This mutant was obtained as follows. First, a plasmid was constructed, pSKDKancya, by inserting within pSKBluescript II vector (Stratagene)—between the XhoI and XbaI sites—the following fragments of DNA: a 1.5 kb fragment of E. coli chromosomal DNA immediately upstream from the cya4 gene, a chloramphenicol-resistant cassette, a 1.5 kb fragment of E. coli chromosomal DNA immediately downstream from the cyaA gene, and a kanamycin-resistant cassette (Karimova, unpublished data). Plasmid pSKDKancya was transformed into the donor strain Hfr KL800 and the resulting transformants were mated with the recipient strain MCI 061 (Streptomycin resistant) for 30 min at 37° C. Aliquots of a mating mixture were plated on MacConkey maltose indicator plates supplemented by Kan (50 μg/ml) and Str (100 μg/ml, to conterselect donor cells). The recipient Kan^{R} and Str^{R} cells were then mixed with a lambda vir phage stock in order to select cya derivatives (E. coli cya cells are resistant to λ) and re-plated on MacConkey maltose. Several λ^{R}, cya derivatives were isolated. None of them harbored the intended cya deletions, but several of the clones obtained exhibited a stable cya phenotype (i.e., they reverted towards Lac⁺ or Mal⁺ phenotypes with a very low frequency). To identify clones, which lost the plasmid, the reisolated recombinants were subsequently plated on LB-Kan and MacConkey maltose indicator plates. One of such cya, Kan^{S}-sensitive clones, BTH99, was kept for further study as it exhibited good complementation capabilities in bacterial two-hybrid assays with standard complementing plasmids (pKT25-zip/pUT18-zip, see FIG. 1 and FIG. 2). The mutation cya-99 of BTH99 is currently under characterization.

BTH101 is a derivative of BTH99 that was created by two successive P1 transductions. First, the wild type lac operon was transduced from FB8, the transductants were selected on minimal plates supplemented by lactose in the presence of 1 mM cAMP. Lac⁺ clones (BTH100) were reisolated and tested for the presence of cya-99 by using MacConkey/lactose indicator plates with and without cAMP. BTH101 strain was made by PI transduction of the wild type allele of ilv (from FB8) into BTH100. The resulting clones were selected on minimal medium without amino acids. One such clone, BTH101, was kept for further characterization.

Plasmids and strains useful for practicing this invention have been deposited at Collection Nationale de Cultures de Microorganismes in Paris, France as follows:

| Plasmid | Deposit Date | Accession No. |
|---|---|---|
| XL-1/pUT18 | Nov. 25, 1998 | 1-2092 |
| XL-1/pUT18C | Nov. 25, 1998 | 1-2093 |
| XL-1/pT25 | Nov. 25, 1998 | 1-2094 |
| XL-1/pKT25 | Nov. 25, 1998 | 1-2095 |
| DHM1 | Sep. 10, 1999 | 1-2310 |
| BTH101 | Sep. 10, 1999 | 1-2309 |
| H. pylon | Dec. 14, 1999 | 1-2367 |
| library | | |
| HGX BHP₃ | | |

Plasmid pKr25 (3445-bp) is a derivative of the low copy vector pSU40 (expressing a kan amycin resistance selectable marker) that encodes the T25 fragment. It was constructed as follows: a 1044-bp Hindlll-EcoRI, fragment of pT25 was first subdloned into pSU40 linearized with HindIII and EcoRI, resulting in pKT25L. pKT25 was generated from pKT25L by deleting a 236-bp NheI-HindIII fragment.

Plasmid pUT18 (3023-bp) is a derivative of the high copy number vector pUC19 (expressing an ampicillin resistance selectable marker and compatible with pT25 or pKT25) that encodes the T18 fragment (amino acids 225 to 399 of CyaA). In a first step, we constructed plasmid pUC19L by inserting a 24bp double-stranded oligonucleotide (5′-ATTCATCGATATAACTAAGTAA-3′ [SEQ ID No. 1]) and its complementary sequence) between the EcoRI and NdeI sites of pUC19. Then, a 534-bp fragment harboring the T18 open reading frame was amplified by PCR (using appropriate primers and pT18 as target DNA) and cloned into pUC19L digested by EcoRI and ClaI (the appropriate restriction sites were included into the PCR primers). In the resulting plasmid, pUT18, the T18 open reading frame is fused in frame downstream of the multicloning site of pUC19. This plasmid is designed to create chimeric proteins in which a heterologous polypeptide is used to the N-terminal end of T18 (see map).

Plasmid pUT18C (3017-bp) is a derivative of pUC19 (expressing an ampicillin resistance selectable marker and compatible with pT25 or pKT25) that encodes the T18 fragment. It was constructed by subeloning the same 534bp PCR-amplified fragment harboring the T18 open reading frame described above into pUC19L linearized by HindIII and PstI (the appropriate restriction sites were included into the PCR primers). In the resulting plasmid, pUT18C, the T18 open reading frame is fused in frame upstream of the multicloning site of pUC19L. This plasmid is designed to create chimeric proteins in which a heterologous polypeptide is fused to the C-terminal end of T18 (see map).

Plasmid pKT25-zip (3556-bp) is a derivative of pKT25 that was constructed by inserting a DNA fragment (CR-amplified using appropriate primers) encoding the leucine zipper region of GCN4 into pKT25 cleaved by KpnI, as described above.

Plasmid pUT18-zip (3125-bp) is a derivative of pUT18 that was constructed by inserting a 114 bp DNA fragment (PCR-amplified using appropriate primers) encoding the leucine zipper region of GCN4 into pUT18 linearized by KpnI and EcoRI.

Plasmid pUT18C-zip (3119-bp) is a derivative of pUT18C that was constructed by inserting the same 114-bp DNA fragment encoding the GCN4 leucine zipper described above into pUT18 linearized by KpnI and EcoRI.

This invention will be described in greater detail with reference to the following examples.

### EXAMPLE 1

To examine the frequency of Lac+ and Mal+ revertants among BTH101 cells, an overnight culture of BTH101 (10 ml) in LB was collected by centrifugation, washed 3 times by B63 medium, and plated on minimal medium with X-gal supplemented by maltose or lactose as unique carbon sources. Frequencies of Lac⁺ and Mal⁺ clones were, respectively, 10⁻⁸ and 10⁻⁹.

### EXAMPLE 2

To test the efficiency of complementation in the bacterial two-hybrid system, the different strains, DHP1, DHM1, and BTH101, were co-transformed with the various couples of plasmid as indicated in FIGS. 1 and 2. The β-galactosidase activity expressed by each co-transformant was determined on liquid culture as previously described (Karimova et al., 1998). Data of FIGS. 1 and 2 correspond to two different sets of totally independent experiments.

### EXAMPLE 3

Vector Construction

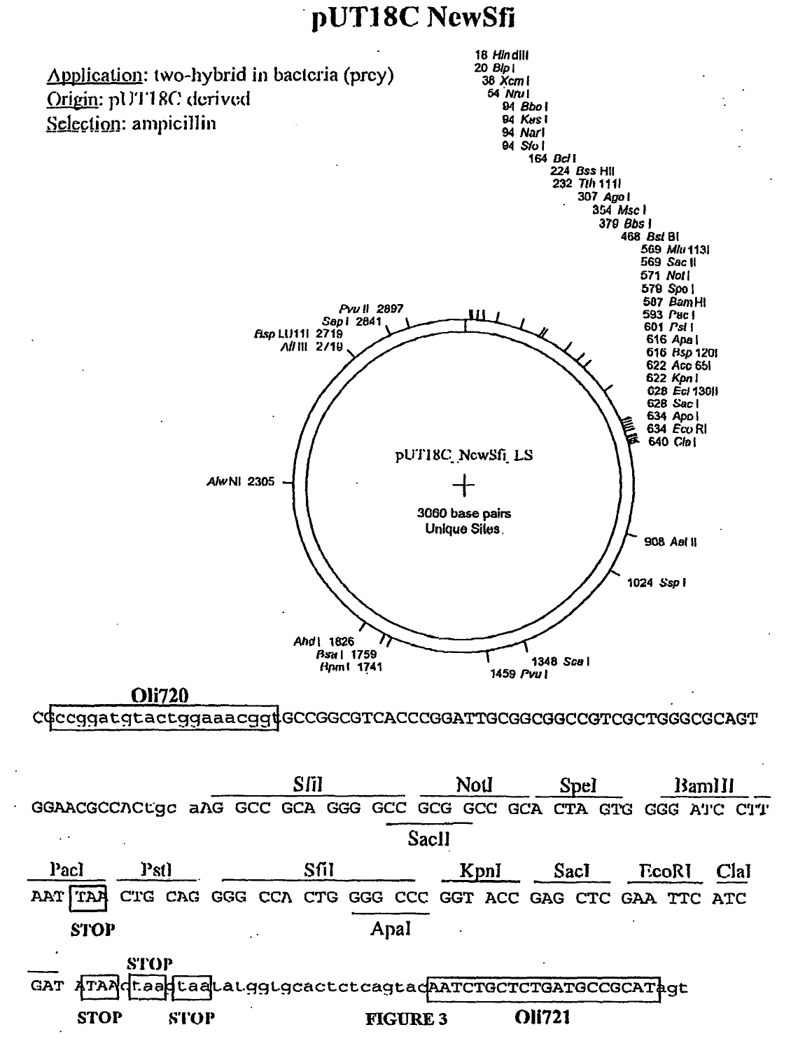

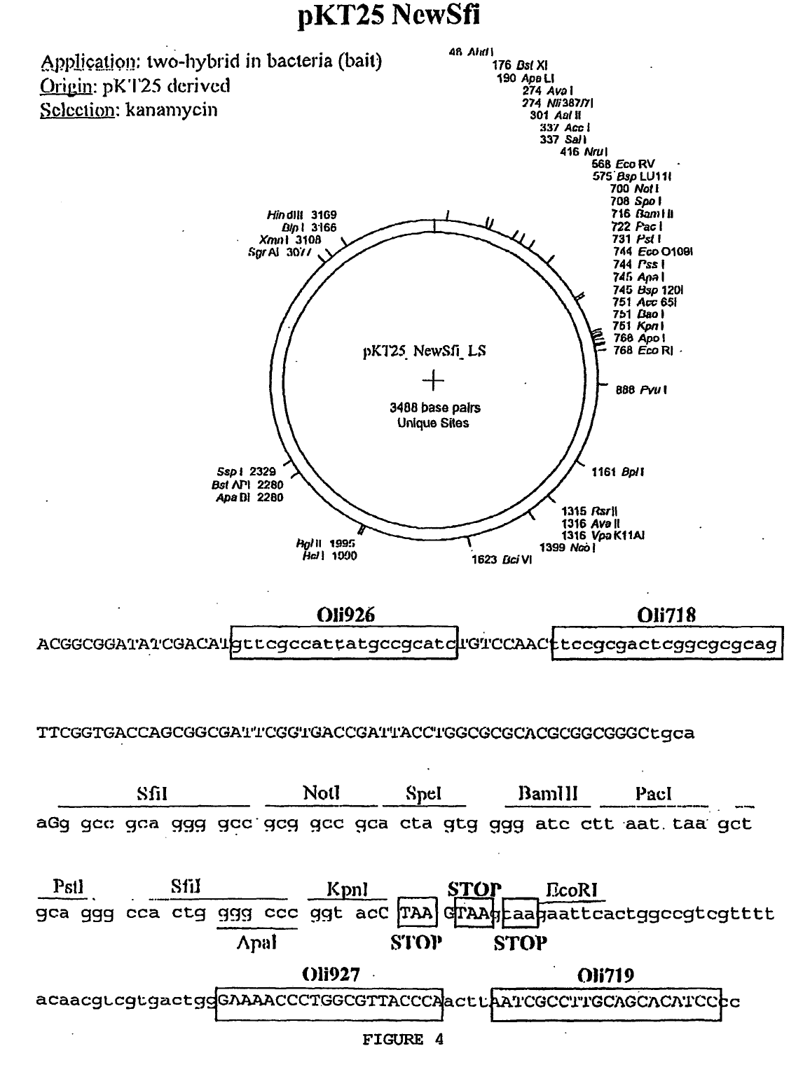

pUT18C is a high-copy number plasmid since it contains the Co1E1 origin of replication. It was, therefore, used as the prey vector. A new multi-cloning site was created by ligating a long oligonucleotide within the KpnI-PstI sites. The sequences of the top and bottom oligonucleotides were as follows: AGGCCGCAGGGGCCGCGGCC GCACTAGTGGGGATCCTTAATTAACTGCAGGGGCCACTGGGGCCCGGTAC [SEQ ID No. 2] and CGGGCCCCAGTGGCCCCTGCAGTTAATRAA GGATCCCCACTAGTGCGGCCGCGGCCCCTGCGGCCTTGCA [SEQ ID No. 3], respectively. Both oligos were annealed and the resulting double-stranded oligo was ligated into previously digested pUT18C vector. pKT25 NewSFI, a derivative of pKT25, was used as the bait vector. It contains a multi-cloning site similar to the one of pUT18C NewSFI and was constructed with the top AGGGCCGCAGGGGCCGCGGC CGCACTAGTGGGGATCCTTAATTAAGCTGCAGGGCCACTGGGGCCCGGTAC [SEQ ID No. 4] and bottom CGGGCCCCAGTGGCCCTGCAGCTTAATTAA GGATCCCCACTAGTGCGGCCGCGGCCCCTGCGGCCCTTGCA [SEQ ID No. 5] oligos. Maps of these two vectors can be found in FIGS. 3 and 4.

7.4×10⁶ independent colonies were recovered after electroporation of the ligation products. Out of the 192 randomly chosen colonies, 85 (44%) contained a plasmid with an insert according to a PCR screening experiment and were subsequently sequenced. Seventy nine sequences were exploitable. 41 (52%) of the insert-containing plasmids were found in the sense orientation. No obvious bias of cloning was observed from the calculation of the start positions of the inserts. The average size of the 79 inserts was not determined.

### EXAMPLE 4

Library Construction

The method used to clone genomic fragments from Helicobacter pylori into pUT18CNS has been described elsewhere (WO 99/28746). Briefly, pUT18CNS was BamHI digested and filled in with a guanosine nucleotide to prevent self-religation of the vector. Nebulized genomic DNA from H. pylori was blunt-ended using a cocktail of Mung Bean Nuclease, T4 Polymerase and Klenow enzymes. Inserts were further ligated with adapters into linearized pUT18CNS plasmid. DH10B (Gibco BRL) electro-competent cells were transformed with ligation products and plated on LB+ ampicillin. One hundred and ninety two colonies were randomly chosen to estimate the actual number of plasmids with inserts and to detect an eventual bias during the cloning step. These colonies were used as template in PCR experiments using the 720 and 721 oligos. PCR products were further sequenced using the same oligos. Sequences were treated with BLAST software. Remaining colonies were harvested and pooled. 20 ml were aliquoted with glycerol or DMSO in 1 ml cryotubes and stored at −80° C. Remaining of the pool was aliquoted in 50 ml Falcon tubes and centrifuged. Supernatants were removed and tubes were stored at −80° C. Library DNA was extracted using a Qiagen Maxi-Prep kit.

### EXAMPLE 5

Bait Construction

pKT25NS was digested with the BamHI and PstI restriction enzymes. The vector was further dephosphorylated using Calf Intestine Phosphatase. The urease accessory protein UreH (HP0067) has been previously used in a genome-wide approach of Helicobacter pylori two-hybrid in yeast screens (WO 99/28746). The insert corresponding to the full-length coding sequence was BamHI-PstI subcloned from pAS2ΔΔ-HP0067 into newly prepared pKT25 NewSFI.

### EXAMPLE 6

Library Screening

DHM1 was used as the cya⁻ host strain. Electro-competent DHM1 cells were transformed with pKT25 NewSFI-HP0067 and plated on LB+kanamycin. pKT25 NewSFI-HP0067 cells were subsequently rendered electro-competent and transformed with 1:1 of diluted H. pylori library (40 ng). Transformed cells were incubated 1 hour at 37° C. after adding 1 ml of SOC medium and were further washed with 1 ml M9 medium. Following a 1/10000^{th} dilution, cells were plated on LB+kanamycin+ampicillin for counting of double-transformants. Remaining of the transformed cells was plated either undiluted on 9 M63+maltose+X-Gal+IPTG+kanamycin+ampicillin plates or diluted 1/10^{th} on 9 plates of the same medium or diluted 1/100^{th} on 10 plates of the same medium.

5×10⁷ double transformants were obtained following transformation of the DHM1/pKT25 NewSFI-HP0067 with 40 ng of library DNA. About 5×10⁶, 5×10⁵ and 5×10⁴ bacteria were, therefore, plated on the M63 screening plates, when no, 1/10^{th} or 1/10th dilutions were performed, respectively. During a 15 day growth period, 18 positive clones were selected, 3 in the 0 dilution condition, 1II in the 1/10^{th} dilution condition, and 4 in the 1/100^{th} dilution condition.

### EXAMPLE 7

Prev Isolation and Identification

Following growth and blue coloration at 30° C., positive clones were further streaked on M63+maltose+X-Gal+IPTG+kanamycin+ampicillin plates to isolate positive clones from the background negative colonies. Plasmid mini-preps (Qiagen) following overnight culture in LB+kanamycin were made to recover the prey plasmids. Prey inserts were further sequenced using the 720 and 721 primers. BLAST homology was performed on these sequences.

All XX positive clones were sequenced. Clone identification is shown in Table 2.

TABLE 2

| Bait | Dilution | Clone | Strand | Pos. | ORF | nt/AUG | Osize | Phase |
|---|---|---|---|---|---|---|---|---|
| HP0067 | 0 | 2 | C | 74170 | HP0069 | 41 | 765 | IF |
| HP0067 | 0 | 3 | C | 74182 | HP0069 | 29 | 765 | IF |
| HP0067 | 0 | 4 | W | 1566571 | HP1493 | 30 | 612 | OOF |
| HP0067 | 10 | 1 | C | 117917 | HP0109 | 308 | 1863 | IF |
| HP0067 | 10 | 2 | W | 692044 | HP0645 | 1681 | 1683 | OOF |
| HP0067 | 10 | 3 | ND | ND | HP1493 | ND | ND | ND |
| HP0067 | 10 | 4 | W | 1566580 | HP1493 | 39 | 612 | OOF |
| HP0067 | 10 | 5 | C | 488358 | HP0466 | 320 | 768 | IF |
| HP0067 | 10 | 7 | C | 74170 | HP0069 | 41 | 765 | IF |
| HP0067 | 10 | 8 | C | 74194 | HP0069 | 17 | 765 | IF |
| HP0067 | 10 | 9 | C | 143584 | HP0132 | 11 | 1368 | IF |
| HP0067 | 10 | 10 | W | 1566580 | HP1493 | 39 | 612 | OOF |
| HP0067 | 10 | 11 | W | 1566576 | HP1493 | 35 | 612 | IF |
| HP0067 | 10 | 12 | C | 74173 | HP0069 | 38 | 765 | IF |
| HP0067 | 100 | 1 | W | 503238 | HP0480 | 611 | 1800 | IF |
| HP0067 | 100 | 2 | W | 364123 | HP0354 | 1574 | 1857 | IF |
| HP0067 | 100 | 3 | C | 1e+06 | HP1422 | 2021 | 2763 | IF |
| HP0067 | 100 | 4 | C | 1261589 | HP1190 | 386 | 1329 | IF |

ND: Not Determined

When sorted according to the position of the first nucleotide of the insert, the results in Table 3 are obtained:

TABLE 3

| Bait | Dilution | Clone | Strand | Pos. | ORF | nt/AUG | Osize | Phase |
|---|---|---|---|---|---|---|---|---|
| HP0067 | 0 | 2 | C | 74170 | HP0069 | 41 | 765 | IF |
| HP0067 | 10 | 7 | C | 74170 | HP0069 | 41 | 765 | IF |
| HP0067 | 10 | 12 | C | 74173 | HP0069 | 38 | 765 | IF |
| HP0067 | 0 | 3 | C | 74182 | HP0069 | 29 | 765 | IF |
| HP0067 | 10 | 8 | C | 74194 | HP0069 | 17 | 765 | IF |
| HP0067 | 10 | 1 | C | 117917 | HP0109 | 308 | 1863 | IF |
| HP0067 | 10 | 9 | C | 143584 | HP0132 | 11 | 1368 | IF |
| HP0067 | 100 | 2 | W | 364123 | HP0354 | 1574 | 1857 | IF |
| HP0067 | 10 | 5 | C | 488358 | HP0466 | 320 | 768 | IF |
| HP0067 | 100 | 1 | W | 503238 | HP0480 | 611 | 1800 | IF |
| HP0067 | 10 | 2 | W | 692044 | HP0645 | 1681 | 1683 | OOF |
| HP0067 | 100 | 4 | C | 1261589 | HP1190 | 386 | 1329 | IF |
| HP0067 | 100 | 3 | C | 1492663 | HP1422 | 2021 | 2763 | IF |
| HP0067 | 0 | 4 | W | 1566571 | HP1493 | 30 | 612 | OOF |
| HP0067 | 10 | 11 | W | 1566576 | HP1493 | 35 | 612 | IF |
| HP0067 | 10 | 4 | W | 1566580 | HP1493 | 39 | 612 | OOF |
| HP0067 | 10 | 10 | W | 1566580 | HP1493 | 39 | 612 | OOF |
| HP0067 | 10 | 3 | ND | ND | HP1493 | ND | ND | ND |

Five out of the 18 clones correspond to another urease accessory protein, ureF (HP0069), which was also found in the corresponding yeast two-hybrid screen of PCT WO 99/28746. Out of these 5 clones, 4 independent fusions (different start positions of the inserts) were found. All of the inserts were found to be in frame with T18. Five out of the 18 clones correspond to BP1493, a predicted coding region of H. pylori, which has no known homology with any other sequence deposited in public gene databases. Out of the 5 clones, at least 3 independent fusions were found. Two of these 3 independent fusions were found out of frame with TI 8. This prey was not found in the corresponding yeast two-hybrid screen (WO 99/28746). It might correspond to a novel protein partner of ureH, which can not be isolated with the yeast two-hybrid technology.

The present invention utilizes the signal amplification system in Escherichia coli described in WO 99/28746, in which the proteins of interest are genetically fused to two complementary fragments of the catalytic domain of Bordetella pertussis adenylate cyclase. B. pertussis produces a calmodulin dependent adenylate cyclase toxin encoded by the cyaA gene.

The catalytic domain is located within the first 400 amino acids of this 1706 residue-long protein.

The catalytic domain can be proteolytically cleaved into two complementary fragments, T25 and T18, that remain associated in the presence of CaM in a fully active ternary complex. In the absence of CaM, the mixture of the two fragments did not exhibit detectable activity suggesting that the two fragments are not able to reassociate to yield basal CaM-independent activity.

The two complementary fragments, T25 and T18, that are both necessary to form an active enzyme, in the presence of CaM when expressed in E. coli as separated entities, are unable to recognize each other and cannot reconstitute a functional enzyme. However, when T25 and T18 are fused to peptides or proteins that are able to interact, heterodimerization of these chimeric polypeptides results in a functional complementation between the adenylate cyclase fragments.

When expressed in an adenylate cyclase deficient E. coli strain (E. coli lacks CaM or CaM-related proteins), the T25 and Ti 8 fragments fused to putative interacting proteins reassociate and lead to cAMP synthesis.

Interaction between a target ligand and a molecule of interest results in functional complementation between the two adenylate cyclase fragments leading to cAMP synthesis, which in turn can trigger the expression of several resident genes. Using this assay, one can select specific clones expressing a protein that interacts with a given target by a simple genetic screening.

The present invention provides a signal amplification system comprising a bacterial multi-hybrid system, and more preferably a two-hybrid system, of at least two chimeric polypeptides containing a first chimeric polypeptide corresponding to a first fragment of an enzyme, and a second chimeric polypeptide corresponding to a second fragment of an enzyme or a modulating substance capable of activating said enzyme wherein the first fragment is fused to a molecule of interest and the second fragment or the modulating substance is fused to a target ligand and wherein the activity of the enzyme is restored by the in vivo interaction between the said molecule of interest and the said target ligand and wherein a signal amplification is generated; and wherein signal amplification is performed in an E. coli strain selected from the group consisting of strain BTH101 having C.N.C.M. Deposit Accession No. I-2309 and strain DHM1 having C.N.C.M. Deposit Accession No. I-2310.

According to one embodiment of the invention, the enzyme can be selected from the group consisting of adenylate cyclase and guanylate cyclase from any origin. Any origin refers to Bordetella species or any other organism that produces this type of enzyme. In one specific illustration, the enzyme is the catalytic domain of Bordetella adenylate cyclase, (CyaA) located within the first 400 amino acid residues of the adenylate cyclase enzyme as described in WO 99/28746.

The present invention also concerns a first fragment and a second fragment, which are any combination of fragments from the same enzyme, which in vitro functionally interact with the natural activator of said enzyme by restoring its activity.

According to one embodiment of the invention the first and the second fragments are selected from the following group described in WO 99/28746.

(a) a fragment T25 corresponding to amino acids 1 to 224 of CyaA and a fragment T18 corresponding to amino acids 225 to 399 of CyaA;

(b) a fragment corresponding to amino acids 1 to 224 of CyaA and a fragment corresponding to amino acids 224 to 384 of CyaA;

(c) a fragment corresponding to amino acids 1 to 137 of CyaA and a fragment corresponding to amino acids 138 to 400 of CyaA;

(d) a fragment corresponding to amino acids 1 to 317 of CyaA and a fragment corresponding to amino acids 318 to 400 of CyaA; and

(e) two fragments from eukaryotic adenylate cyclase in association with molecules, such as, G protein and forskolin.

The first and the second fragments can be a fragment T25 corresponding to amino acids 1 to 224 of Bordetella pertussis CyaA and a fragment T18 corresponding to amino acids 225 to 399 of Bordetella pertussis CyaA as described in WO 99/28746. In one embodiment, the first fragment is a mutated fragment of the catalytic domain of Bordetella adenylate cyclase (CyaA).

The modulating substance can be a natural activator, or a fragment thereof, of the enzyme. The natural activator can be the calmodulin (CaM), or a fragment thereof, and the first fragment can be mutated compared to the wild type enzyme. The fragment of calmodulin can be about 70 amino acids long, corresponding preferentially, to residues 77 to 148 of mammalian calmodulln, as described in WO 99/28746.

The present invention also concerns a method of selecting a molecule of interest which is capable of binding to target ligand wherein the interaction between the said molecule of interest and the said target ligand is detected with a signal amplification system of the invention, by means of generating a signal amplification and triggering transcriptional activation or repression, wherein the method of selecting the molecule of interest is performed in an E. coli strain selected from the group consisting of strain BTH101 having C.N.C.M. Deposit Accession No. I-2309 and strain DHM1 having C.N.C.M. Deposit Accession No. I-2310.

In a first embodiment, the signal amplification system according to the invention comprises a bacterial multi-hybrid system of at least two distinct fragments of an enzyme, whose enzymatic activity is restored by the interaction between the said molecule of interest and the said target ligand.

In a second embodiment, the signal amplification system according to the invention comprises bacterial multi-hybrid system of at least a first fragment of an enzyme and a modulating substance, whose activity is restored by the interaction between the said molecule of interest and the said target ligand.

The signal amplification system according to the invention comprises a bacterial multi-hybrid system as described in WO 99/28746.

In the method of selecting a molecule of interest of the invention, the interaction between the molecule of interest and the target ligand is detected, by means of signal amplification which triggers transcriptional activation, and is quantified by measuring the synthesis of the signaling molecule or the expression of the reporter gene.

The signal amplification corresponds to the production of a signaling molecule. This signaling molecule is any molecule capable of leading to a signaling cascade reaction. In one embodiment the signaling molecule corresponds to the synthesis of cAMP. In another embodiment the signaling molecule corresponds to the synthesis of cGMP.

The transcriptional activation leads to a reporter gene expression; the reporter gene is selected from the group consisting of gene coding for nutritional marker, such as lactose or maltose; gene conferring resistance to antibiotics such as ampicillin, chloramphenicol, kanamycin, or tetracyclin; a gene encoding for a toxin; a color marker, such as, fluorescent marker of the type of the Green Fluorescent Protein (GFP); a gene encoding phage receptor proteins or a fragment thereof, such as phage λ receptor, lamB, and any other gene giving a selectable phenotype.

In one embodiment, cAMP, upon binding to CAP, is able to activate the transcription of catabolic operons, allowing the bacteria to ferment carbohydrates, such as maltose or lactose, and to express the phage λ receptor, protein LamB, which could serve as a marker at the bacterial surface. This signal amplification system comprising this bacterial multi-hybrid system using BTH101 or DHM1 is able to reveal, for example, interactions between small peptides (GCN4 leucine zipper), bacterial (tyrosyl tRNA synthetase), or eukaryotic proteins (yeast Prp11/Prp21 complex).

Accordingly, specific reporter cassettes in which any gene of interest is fused to a cAMP/CAP dependent promoter can be designed. Thus, to facilitate the screening and the selection of complex libraries, the construction of such a simple selection system using an antibiotic resistance gene can be performed.

The reporter gene could be a toxin, not naturally present in bacteria, under the control of a cAMP/CAP-dependent promoter. This could be particularly useful to search for chemical compounds or mutations that abolish a given interaction between the target ligand and a molecule of interest. According to this construction, when association between the target ligand and a molecule of interest takes place, cAMP will be produced, the expression of the toxin gene will be switched on, and the cells will be killed. A substance capable of stimulating or inhibiting the interaction between the .target ligand and the molecule of interest and that abolishes interaction will shut down toxin gene expression and will enable the cells to grow. An easy selection for substances that abolish interaction between the target ligand and the molecule of interest is resistance to phage λ. The phage receptor, the LamB protein, is the product of the lamB gene, which is part of the maltose regulon, therefore its expression requires cAMP. Substances that abolish interaction between the target ligand and the molecule of interest will abrogate cAMP synthesis and bacteria will become resistant to phage λ. As a result, the cells will grow.

Another selection scheme for compounds or mutations that abolish a given interaction could be designed by constructing a strain that harbors a selectable marker (i.e. a gene conferring resistance to antibiotics such as ampicillin chloramphenicol, kanamycin, tetracyclin, etc.) under the transcriptional control of a promoter that is repressed by cAMP/CAP. Such cAMP/CAP repressed promoter can be engineered by introducing a synthetic CAP binding site within the promotor region as shown by Morita et al. (Morita T, Shigesada K., Kimizuka F., Aiba H. (1988), “Regulatory effect of a synthetic CRP recognition sequence placed downstream of a promoter” Nucleic Acids Res. 16:7315-32).

The International Patent Applications no WO 96/23898 (Thastrup O. et al.) and no WO 97/11094 (Thastrup O. et al.), respectively, relating to a method of detecting biologically active substances as Green Fluorescent Protein (GFP), and the International Patent Application no WO 97/07463 (Chalfie M. et al.) describing the uses of GFP, are herein incorporated by reference, and a novel variant of GFP.

The target ligand according to the invention is selected from the group consisting of protein, peptide, polypeptide, receptor, ligand, antigen, antibody, DNA binding protein, glycoprotein, lipoprotein and recombinant protein.

According to the method of selecting a molecule of interest, the molecule of interest is capable of interacting with the target ligand and possibly of binding to said target ligand. In a specific embodiment of the method of selecting a molecule of interest, the molecule of interest is a mutant molecule compared to the known wild type molecule, and said molecule of interest is tested for its capacity of interacting with the target ligand.

The present invention includes a molecule of interest identified by the method of selecting a molecule of interest according to the present invention. The present invention further includes a molecule of interest corresponding to a polynucleotide capable of expressing a molecule, which interacts with a fused target ligand coupled with an enzyme or a fragment thereof.

In a preferred embodiment, the selection of the molecule of interest of the invention is performed in strain DHM1. In another preferred embodiment, the selection of the molecule of interest of the invention is performed in strain BTH101.

The present invention also concerns a kit for selecting molecule of interest, wherein said kit comprises: (a) a signal amplification system of the invention; (b) a bacterial strain deficient in endogenous adenylate cyclase selected from the group consisting of strain BTH101 having C.N.C.M. Deposit Accession No. I-2309 and strain DHMI having C.N.C.M. Deposit Accession No. 1-2310; (c) a medium allowing the detection of the complementation selected from the group consisting of indicator or selective medium as minimal medium supplemented with lactose or maltose as unique carbon source, medium with antibiotics, medium to visualize fluorescence, luminescense, conventional medium, such as xGAL medium, and medium which allows the sorting by the presence of the phage receptor.

In another embodiment, the invention concerns a kit for selecting molecule of interest, wherein said kit comprises: (a) a signal amplification system of the invention wherein the molecule of interest is a mutant molecule compared to the known wild type molecule; (b) a signal amplification system of the invention wherein the molecule of interest is the known wild type molecule as the control; (c) a bacterial strain deficient in endogenous adenylate cyclase selected from the group consisting of strain BTH101 having C.N.C.M. Deposit Accession No. 1-2309 and strain DHM1 having C.N.C.M. Deposit Accession No. 1-2310; (d) a medium allowing the detection of the complementation selected from the group consisting of indicator or selective medium as minimal medium supplemented with lactose or maltose as unique carbon source, medium with antibiotics, medium to visualize fluorescence, luminescence, conventional medium, such as xGAL medium, and medium which allows the sorting by the presence of the phage receptor for each signal amplification system; (e) means for detecting whether the signal amplification system with the mutant molecule is enhanced or inhibited with respect to the signal amplification system with wild type.

The present invention also concerns a method of screening for substance capable of stimulating or inhibiting the interaction between a target ligand and a molecule of interest wherein respectively the stimulating or the inhibiting activity is detected with a signal amplification system of the invention, by means of generating an amplification and respectively of triggering or of abolishing transcriptional activation wherein said signal amplification and said triggered or abolished transcriptional activation are compared with those obtained from an identical signal amplification system without any substance and wherein the method of screening for the substance is performed in an E. coli strain selected from the group consisting of strain BTH101 having C.N.C.M. Deposit Accession No. 1-2309 and strain DEM1 having C.N.C.M. Deposit Accession No. I-2310.

In a preferred embodiment, the method of screening for substance capable of stimulating or inhibiting the interaction between a target ligand and a molecule of interest of the invention is characterized by the signal amplification system that comprises a bacterial multi-hybrid system of at least two distinct fragments of an enzyme, whose enzymatic activity is restored by the interaction between the said molecule of interest and the said target ligand. In another preferred embodiment, the method of screening for substance capable of stimulating or inhibiting the interaction between a target ligand and a molecule of interest of the invention is characterized by the signal amplification system that comprises a bacterial multi-hybrid system of at least a first fragment of an enzyme and a modulating substance, whose activity is restored by the interaction between the said molecule of interest and the said target ligand.

In the method of screening for substance capable of stimulating the interaction between a target ligand and a molecule of interest of the invention, the signal amplification corresponds to the production of a signaling molecule.

In the method of screening for substance capable of inhibiting the interaction between a target ligand and a molecule of interest of the invention, the signal amplification corresponding to the production of a signaling molecule is blocked or partially abolished.

In the method of screening for substance capable of stimulating the interaction between a target ligand and a molecule of interest of the invention, the transcriptional activation leads to a reporter gene expression.

In the method of screening for substance capable of inhibiting the interaction between a target ligand and a molecule of interest of the invention, the transcriptional activation leading to a reporter gene expression is blocked or partially abolished.

In the method of screening for substance capable of stimulating or inhibiting the interaction between a target ligand and a molecule of interest of the invention, the target ligand is selected from the group consisting of receptor, ligand, antigen, antibody, DNA binding protein, glycoprotein and lipoprotein, and the substance is selected from the group consisting of protein, glycoprotein, lipoprotein, ligand and any other drug having stimulating or inhibitory affinity.

In the method of screening for substance capable of stimulating or inhibiting the interaction between a target ligand and a molecule of interest of the invention, the signaling molecule corresponds to the synthesis of cAMP or to the synthesis of cGMP.

In the method of screening of the invention, the reporter gene is selected from the group consisting of gene coding for nutritional marker such as lactose, maltose; gene conferring resistance to antibiotics such as ampicillin, kanamycin or tetracyclin; gene encoding for toxin; color marker such as fluorescent marker of the type of the Green Fluorescent Protein (GFP); gene encoding for phage receptor proteins or fragment thereof such as phage λ receptor, lamB and any other gene giving a selectable phenotype.

In a preferred embodiment, in the method of screening of the invention, the molecule of interest is a mutant molecule compared to the known wild type molecule and said molecule of interest is tested for its capacity of interacting with the target ligand.

In a preferred embodiment, the screening for substance is performed in strain DHM1. In another preferred embodiment, the screening for substance is performed in strain BTH101.

The present invention also concerns a kit for screening for substance capable of stimulating or inhibiting the interaction between a target ligand and a molecule of interest, wherein said kit comprises (a) a signal amplification system of the invention with the substance capable of stimulating or inhibiting the interaction between a target ligand and a molecule of interest; (b) a signal amplification system of the invention without any substance as the control; (c) a bacterial strain deficient in endogenous adenylate cyclase selected from the group consisting of strain BTH101 having C.N.C.M. Deposit Accession No. I-2309 and strain D[M1 having C.N.C.M. Deposit Accession No. I-2310; (d) a medium allowing the detection of the complementation selected from the group consisting of indicator plate or selective medium as minimal medium supplemented with lactose or maltose as unique carbon source, medium with antibiotics, medium to visualize fluorescence, conventional medium and medium which allows the sorting by the presence of the phage receptor and; (e) means for detecting whether the signal amplification system with the substance is enhanced or inhibited with respect to the signal amplification system without any substance.

Functional analysis of B. pertussis adenylate cyclase activity can be easily monitored in an E. coli strain deficient in endogenous adenylate cyclase. In E. coli, cAMP bound to the transcriptional activator, CAP (catabolite activator protein), is a pleiotropic regulator of the expression of various genes, including genes involved in the catabolism of carbohydrates, such as lactose or maltose (Ullmann, A. & Danchin, A. (1983) in Advances in Cyclic Nucleotide Research (Raven Press, New York), Vol. vol. 15, pp. 1-53). Hence, E. coli strains lacking cAMP are unable to ferment lactose or maltose. When the entire catalytic domain of CyaA (amino acids 1 to 399) is expressed in E. coli cya under the transcriptional and translational control of lacZ (plasmid pDIA5240), its calmodulin-independent residual activity is sufficient to complement an adenylate cyclase deficient strain and to restore its ability to ferment lactose or maltose (Ladant, D., Glaser, P. & Ullmann, A. (1992) J. Biol. Chem. 267, 2244-2250). This can be scored either on indicator plates (i.e. LB-X-Gal or MacConkey media supplemented with maltose) or on selective media (minimal media supplemented with lactose or maltose as unique carbon source).

The present invention further relates to the a molecule of interest identified by the methods of the invention.

The present invention also concerns the substance capable of stimulating or inhibiting the interaction between a target ligand and a molecule of interest identified by the method of the invention.

The present invention also concerns the signal amplification system of the invention, wherein the bacterial multi-hybrid system contains: (a) a first chimeric polypeptide corresponding to a first fragment a of an enzyme; (b) a second chimeric polypeptide corresponding to a second fragment of an enzyme or a modulating substance capable of activating said enzyme; (c) a substance capable of stimulating or inhibiting the interaction between a target ligand and a molecule of interest, wherein the first fragment is fused to a molecule of interest and the second fragment or the modulating substance is fused to a target ligand and wherein the activity of the enzyme is restored by the interaction between the said molecule of interest and the said target ligand and wherein a signal amplification is generated; and a bacterial strain deficient in endogenous adenylate cyclase selected from the group consisting of strain BTH101 having C.N.C.M. Deposit Accession No. 1-2309 and strain DHM1 having C.N.C.M. Deposit Accession No. I-2310.

The present invention also concerns a DNA library containing a collection of vectors transformed in a bacterial multi-hybrid system, wherein each vector contains a polynucleotide coding for the molecule of interest fused to a polynucleotide encoding for a first or second fragment of an enzyme; the polynucleotide is selected from the group consisting of cDNA, RNA, genomic DNA, mitochondrial DNA. In a preferred embodiment, the DNA library of the invention is the H. pylori DNA library having C.N.C.M. Deposit Accession No. I-2367.

The invention further concerns the strain BTH101 having C.N.C.M. Deposit Accession No. I-2309 and the strain DHM1 having C.N.C.M. Deposit Accession No. I-2310.

In summary, this invention provides the construction and selection of two new E. coli cya strains that overcome the limitations of the DHPB1 strain described in published PCT application WO 99/28746. The new strains, DHM1 and BTH101, exhibit a stable Cya⁻ phenotype: DHM1 has an internal deletion of about 200 bp within the cya gene, BTH101 harbors an uncharacterized mutation within the cya gene which is most likely a deletion, as no Cya+revertant could be isolated from this strain.

Functional complementation between standard hybrid proteins appeared to be as efficient in BTH101 as it is in DHP1. Although less efficient than in DHP1, functional complementation between the same set of standard hybrid proteins in DHM1 is still higher than in all other E. coli cya⁻ strains tested.

These two strains were used as recipient cells to perform a genome-wide library screen of Helicobacter pylori fragments or complete open reading frames, searching for polypeptides that can interact with defined “bait” proteins. These results show that, using these new strains, the bacterial two-hybrid screen can be applied for large scale analysis of protein-protein interactions.

As it appears from the teachings of the specification, the invention is not limited in scope to one or several of the above detailed embodiments; the present invention also embraces all the alternatives that can be performed by one skilled in the same technical field, without deviating from the subject or from the scope of the instant invention.

References

The following publications are cited herein. The entire disclosure of each publication is relied upon and incorporated by reference herein.

1. Glaser, P., Roy, A., A. Danchin, 1989, J. Bacteriol., 171: 5176-5178. Molecular characterization of two cya mutations, cya-854 and cyaR1.

2. Karimova, G., J. Pidoux, A. Ullmann and D. Ladant, 1998, Proc. Natl. Acad. Sci. U.S.A. 95:5752-5756. A bacterial two-hybrid system based on a reconstituted signal transduction pathway.

3. Miller, J. H., 1972, Experiments in Molecular Genetics.

4. Wanner, B. L., 1986, J. Mol. Biol., 191: 39-58. Novel regulatory mutants of the phosphate regulon in Escherichia coli K-12.

5. Sancar, A., Rupp, W. D., 1979. Physical map of the recA gene. Proc. Natl. Acad. Sci. U.S.A. 76:3144-3148.

1. A signal amplification system comprising a bacterial multi-hybrid system of at least two chimeric polypeptides containing:
(a) a first chimeric polypeptide corresponding to a first fragment of an enzyme;
(b) a second chimeric polypeptide corresponding to a second fragment of an enzyme or a modulating substance capable of activating said enzyme,
wherein the first fragment is fused to a molecule of interest and the second fragment or the modulating substance is fused to a target ligand and wherein the activity of the enzyme is restored by the in vivo interaction between the said molecule of interest and the said target ligand and wherein a signal amplification is generated; and
wherein signal amplification is performed in an E. coli strain selected from the group consisting of strain BTH101 having C.N.C.M. Deposit Accession No. I-2309 and strain DHM1 having C.N.C.M. Deposit Accession No. 1-2310.

2. The signal amplification system according to claim 1, wherein the enzyme is an enzyme selected from the group consisting of adenylate cyclase and guanylate cyclase from any origin.

3. The signal amplification system according to claim 2, wherein the enzyme is the catalytic domain of Bordetella adenylate cyclase (CyaA), located within the first 400 amino acid residues of the adenylate cyclase enzyme.

4. The signal amplification system according to claim 3, wherein the first and the second fragments are any combination of fragments from the same enzyme which in vitro functionally interact with the natural activator of said enzyme by restoring its activity.

5. The signal amplification system according to claim 4, wherein the first and the second fragments are selected from the group consisting of:
(a) a fragment T25 corresponding to amino acids 1 to 224 of CyaA and a fragment T18 corresponding to amino acids 225 to 399 of CyaA;
(b) a fragment corresponding to amino acids 1 to 224 of CyaA and a fragment corresponding to amino acids 224 to 384 of CyaA;
(c) a fragment corresponding to amino acids 1 to 137 of CyaA and a fragment corresponding to amino acids 138 to 400 of CyaA;
(d) a fragment corresponding to amino acids 1 to 317 of CyaA and a fragment corresponding to amino acids 318 to 400 of CyaA;
(e) two fragments from eukaryotic adenylate cyclase in association with molecules such as G protein, forskolin.

6. The signal amplification system according to claim 4 or 5, wherein the first and the second fragments are a fragment T25 corresponding to amino acids 1 to 224 of Bordetella pertussis CyaA and a fragment T18 corresponding to amino acids 225 to 399 of Bordetella pertussis CyaA.

7. The signal amplification system according to any one of the claims 1 to 3, wherein the modulating substance is a natural activator, or a fragment thereof, of the enzyme.

8. The signal amplification system according to claim 7, wherein the natural activator is the calmodulin (CaM), or a fragment thereof, and said first fragment is mutated compared to the wild type enzyme.

9. The signal amplification system according to claim 8, wherein the first fragment is a mutated fragment of the catalytic domain of Bordetella adenylate cyclase (CyaA).

10. A DNA library containing a collection of vectors transformed in a bacterial multi-hybrid system, wherein each vector contains a polynucleotide coding for the molecule of interest fused to a polynucleotide encoding for a first or second fragment of an enzyme.

11. DNA library according to claim 10, wherein the polynucleotide is selected from the group consisting of cDNA, RNA, genomic DNA, mitochondrial DNA.

12. DNA library according to claims 10 and 11, wherein the H. pylori DNA library has C.N.C.M. Deposit Accession No. I-2367.

13. A method of selecting a molecule of interest which is capable of binding to target ligand wherein the interaction between the said molecule of interest and the said target ligand is detected with a signal amplification system according to any one of the claims 1 to 9, by means of generating a signal amplification and triggering transcriptional activation or repression, wherein the method of selecting the molecule of interest is performed in an E. coli strain selected from the group consisting of strain BTH101 having C.N.C.M. Deposit Accession No. I-2309 and strain DHM1 having C.N.C.M. Deposit Accession No. I-2310.

14. The method of selecting a molecule of interest according to claim 13, wherein the signal amplification corresponds to the production of a signaling molecule.

15. The method of selecting a molecule of interest according to claim 13, wherein the transcriptional activation leads to a reporter gene expression.

16. The method of selecting a molecule of interest according to any one of claims 13 to 15, wherein the signal amplification system comprises a bacterial multi-hybrid system of at least two distinct fragments of an enzyme, whose enzymatic activity is restored by the interaction between the said molecule of interest and the said target ligand.

17. The method of selecting a molecule of interest according to any one of claims 13 to 15, wherein the signal amplification system comprises bacterial multi-hybrid system of at least a first fragment of an enzyme and a modulating substance, whose activity is restored by the interaction between the said molecule of interest and the said target ligand.

18. The method of selecting a molecule of interest according to any one of claims 13 to 17, wherein the target ligand is selected from the group consisting of protein, peptide, polypeptide, receptor, ligand, antigen, antibody, DNA binding protein, glycoprotein, lipoprotein and recombinant protein.

19. The method of selecting a molecule of interest according to any one of claims 13 to 18, wherein the molecule of interest is capable of interacting with the target ligand and possibly of binding to said target ligand.

20. The method of selecting a molecule of interest according to any one of claims 13 to 19, wherein the interaction between the molecule of interest and the target ligand is detected, by means of signal amplification which triggers transcriptional activation, and is quantified by measuring the synthesis of the signaling molecule or the expression of the reporter gene.

21. The method of selecting a molecule of interest according to any one of claims 13 to 20, wherein the signaling molecule corresponds to the synthesis of cAMP.

22. The method of selecting a molecule of interest according to claim 16, wherein the signaling molecule corresponds to the synthesis of cGMP.

23. The method of selecting a molecule of interest according to claim 15, wherein the reporter gene is selected from the group consisting of gene coding for nutritional marker such as lactose, maltose; gene conferring resistance to antibiotics such as ampicillin, kanamycin or tetracyclin; gene encoding for toxin; color marker such as fluorescent marker of the type of the Green Fluorescent Protein (GFP); gene encoding for phage receptor proteins or fragment thereof such as phage λ receptor, lamB and any other gene giving a selectable phenotype.

24. The method of selecting a molecule of interest according to any one of claims 13 to 23, wherein the molecule of interest is a mutant molecule compared to the known wild type molecule and said molecule of interest is tested for its capacity of interacting with the target ligand.

25. The method of selecting a molecule of interest according to any one of claims 13 to 25, wherein the selection is performed in strain DHM1.

26. The method of selecting a molecule of interest according to any one of claims 13 to 25 wherein the selection is performed in strain BTH101.

27. A kit for selecting molecule of interest, wherein said kit comprises:
(a) a signal amplification system according to any one of claims 1 to 9;
(b) a bacterial strain deficient in endogenous adenylate cyclase selected from the group consisting of strain BTH101 having C.N.C.M. Deposit Accession No. I-2309 and strain DHM1 having C.N.C.M. Deposit Accession No. I-2310;
(c) a medium allowing the detection of the complementation selected from the group consisting of indicator or selective medium as minimal medium supplemented with lactose or maltose as unique carbon source, medium with antibiotics, medium to visualize fluorescence, luminescense, conventional medium, such as xGAL medium, and medium which allows the sorting by the presence of the phage receptor.

28. A kit for selecting molecule of interest, wherein said kit comprises:
(a) a signal amplification system according to any one of claims 1 to 9 wherein the molecule of interest is a mutant molecule compared to the known wild type molecule;
(b) a signal amplification system according to any one of claims 1 to 9 wherein the molecule of interest is the known wild type molecule as the control;
(c) a bacterial strain deficient in endogenous adenylate cyclase selected from the group consisting of strain BTH101 having C.N.C.M. Deposit Accession No. I-2309 and strain DHM1 having C.N.C.M. Deposit Accession No. I-2310;
(d) a medium allowing the detection of the complementation selected from the group consisting of indicator or selective medium as minimal medium supplemented with lactose or maltose as unique carbon source, medium with antibiotics, medium to visualize fluorescence, luminescence, conventional medium, such as xGAL medium, and medium which allows the sorting by the presence of the phage receptor for each signal amplification system;
(e) means for detecting whether the signal amplification system with the mutant molecule is enhanced or inhibited with respect to the signal amplification system with wild type.

29. A method of screening for substance capable of stimulating or inhibiting the interaction between a target ligand and a molecule of interest wherein respectively the stimulating or the inlubiting activity is detected with a signal amplification system according to any one of the claims 1 to 9, by means of generating an amplification and respectively of triggering or of abolishing transcriptional activation;
wherein said signal amplification and said triggered or abolished transcriptional activation are compared with those obtained from an identical signal amplification system without any substance; and
wherein the method of screening for the substance is performed in an E. coli strain selected from the group consisting of strain BTH101 having C.N.C.M. Deposit Accession No. I-2309 and strain DHM1 having C.N.C.M. Deposit Accession No. I-2310.

30. The method of screening for substance capable of stimulating or inhibiting the interaction between a target ligand and a molecule of interest according to claim 29, wherein the signal amplification system comprises a bacterial multi-hybrid system of at least two distinct fragments of an enzyme, whose enzymatic activity is restored by the interaction between the said molecule of interest and the said target ligand.

31. The method of screening for substance capable of stimulating or inhibiting the interaction between a target ligand and a molecule of interest according to claim 29, wherein the signal amplification system comprises a bacterial multi-hybrid system of at least a first fragment of an enzyme and a modulating substance, whose activity is restored by the interaction between the said molecule of interest and the said target ligand.

32. The method of screening for substance capable of stimulating the interaction between a target ligand and a molecule of interest according to any one of claims 29 to 31, wherein the signal amplification corresponds to the production of a signaling molecule.

33. The method of screening for substance capable of inhibiting the interaction between a target ligand and a molecule of interest according to any one of claims 29 to 31, wherein the signal amplification corresponding to the production of a signaling molecule is blocked or partially abolished.

34. The method of screening for substance capable of stimulating the interaction between a target ligand and a molecule of interest according to any one of claims 29 to 32, wherein the transcriptional activation leads to a reporter gene expression.

35. The method of screening for substance capable of inhibiting the interaction between a target ligand and a molecule of interest according to any one of claims 29 to 31 and to claim 33, wherein the transcriptional activation leading to a reporter gene expression is blocked or partially abolished.

36. The method of screening for substance capable of stimulating or inhibiting the interaction between a target ligand and a molecule of interest according to any one of claims 29 to 35, wherein the target ligand is selected from the group consisting of receptor, ligand, antigen, antibody, DNA binding protein, glycoprotein and lipoprotein.

37. The method of screening for substance capable of stimulating or inhibiting the interaction between a target ligand and a molecule of interest according to any one of claims 29 to 36, wherein the substance is selected from the group consisting of protein, glycoprotein, lipoprotein, ligand and any other drug having stimulating or inhibitory affinity.

38. The method of screening for substance capable of stimulating or inhibiting the interaction between a target ligand and a molecule of interest according to claim 32 or 33, wherein the signaling molecule corresponds to the synthesis of cAMP.

39. The method of screening for substance capable of stimulating or inhibiting the interaction between a target ligand and a molecule of interest according to claim 32 or 33, wherein the signaling molecule corresponds to the synthesis of cGMP.

40. The method of screening for substance capable of stimulating or inhibiting the interaction between a target ligand and a molecule of interest according to claim 34 or 35, wherein the reporter gene is selected from the group consisting of gene coding for nutritional marker such as lactose, maltose; gene conferring resistance to antibiotics such as ampicillin, kanamycin or tetracyclin; gene encoding for toxin; color marker such as fluorescent marker of the type of the Green Fluorescent Protein (GFP); gene encoding for phage receptor proteins or fragment thereof such as phage λ receptor, lamB and any other gene giving a selectable phenotype.

41. The method of screening for substance capable of stimulating or inhibiting the interaction between a target ligand and a molecule of interest according to any one of claims 29 to 40, wherein the molecule of interest is a mutant molecule compared to the known wild type molecule and said molecule of interest is tested for its capacity of interacting with the target ligand.

42. The method of screening for substance capable of stimulating or inhibiting the interaction between a target ligand and a molecule of interest according to any one of claims 29 to 40, wherein the screening is performed in strain DEM1.

43. The method of screening for a substance capable of stimulating or inhibiting the interaction between a target ligand and a molecule of interest according to any one of claims 29 to 41, wherein the screening is performed in strain BTH101.

44. A kit for screening for substance capable of stimulating or inhibiting the interaction between a target ligand and a molecule of interest, wherein said kit comprises:
(a) a signal amplification system according to any one of claims 1 to 9 with the substance capable of stimulating or inhibiting the interaction between a target ligand and a molecule of interest;
(b) a signal amplification system according to any one of claims 1 to 9 without any substance as the control;
(c) a bacterial strain deficient in endogenous adenylate cyclase selected from the group consisting of strain BTH101 having C.N.C.M. Deposit Accession No. I-2309 and strain DHM1 having C.N.C.M. Deposit Accession No. I-2310;
(d) a medium allowing the detection of the complementation selected from the group consisting of indicator plate or selective medium as minimal medium supplemented with lactose or maltose as unique carbon source, medium with antibiotics, medium to visualize fluorescence, conventional medium and medium which allows the sorting by the presence of the phage receptor and;
(e) means for detecting whether the signal amplification system with the substance is enhanced or inhibited with respect to the signal amplification system without any substance.

45. A molecule of interest identified by the method of any one of the claims 13 to 25.

46. A substance capable of stimulating or inhibiting the interaction between a target ligand and a molecule of interest identified by the method of any one of the claims 29 to 43.

47. The signal amplification system according to any one of the claims 1 to 9, wherein the bacterial multi-hybrid system contains:
(a) a first chimeric polypeptide corresponding to a first fragment a of an enzyme;
(b) a second chimeric polypeptide corresponding to a second fragment of an enzyme or a modulating substance capable of activating said enzyme;
(c) a substance capable of stimulating or inhibiting the interaction between a target ligand and a molecule of interest, wherein the first fragment is fused to a molecule of interest and the second fragment or the modulating substance is fused to a target ligand and wherein the activity of the enzyme is restored by the interaction between the said molecule of interest and the said target ligand and wherein a signal amplification is generated; and
a bacterial strain deficient in endogenous adenylate cyclase selected from the group consisting of strain BTH101 having C.N.C.M. Deposit Accession No. I-2309 and strain DHM1 having C.N.C.M. Deposit Accession No. I-2310.

48. Strain BTH101 having C.N.C.M. Deposit Accession No. I-2309.

49. Strain DHM1 having C.N.C.M. Deposit Accession No. I-2310.
